(19) United States
(12) Patent Application Publication
(10) Pub. No.: US 2014/0135357 A1
(43) Pub. Date: May 15, 2014

(54) DOSE REGIME FOR CAMPTOTHECIN DERIVATIVES

(71) Applicants: Taiwan Liposome Company, Ltd., Taipei City (TW); TLC Biopharmaceuticals, Inc., South San Francisco, CA (US)

(72) Inventors: Yun-Long TSENG, Taoyuan City (TW); Keelung HONG, South San Francisco, CA (US); Min-Wen KUO, Taipei City (TW)

(73) Assignees: Taiwan Liposome Company, Ltd., Taipei City (TW); TLC Biopharmaceuticals, Inc., South San Francisco, CA (US)

(21) Appl. No.: 14/077,353

(22) Filed: Nov. 12, 2013

Related U.S. Application Data
(60) Provisional application No. 61/725,455, filed on Nov. 12, 2012.

(51) Int. Cl. A61K 31/4745; A61K 47/24; C07D 491/22

(52) U.S. Cl. CPC A61K 31/4745; C07D 491/22; A61K 47/24
USPC 514/283

(57) ABSTRACT

The present invention is directed to a method of inhibiting cancer cell growth, comprising administering a pharmaceutical composition to a subject in need thereof. The pharmaceutical composition comprises at least one camptothecin derivative or a pharmaceutically acceptable salt thereof; and at least one PEG phospholipid, and provides a sustained release of topotecan as an active ingredient.

### CROSS REFERENCE TO RELATED APPLICATIONS

This application claims the benefit of U.S. Application No. 61/725,455, filed Nov. 12, 2012, the entire disclosure of which is incorporated herein by reference. This application is related to International Patent Application No. PCT/US2012/63447 filed Nov. 2, 2012, which claims the benefit of U.S. Provisional Patent Application No. 61/555,084 filed Nov. 3, 2011. The contents of each of the above filings are incorporated herein by reference.

### TECHNOLOGY FIELD

The present invention relates to methods of inhibiting cancer cell growth by administering a pharmaceutical composition comprising at least one camptothecin derivative; or a pharmaceutically acceptable salt of said derivative; and at least one polyethylene glycol (PEG) conjugated phospholipid, resulting in a sustained release of topotecan to said subject over a period of at least about 8 hours.

### BACKGROUND OF THE INVENTION

Camptothecin ((S)-4-ethyl-4-hydroxyl-1H-pyrano-[3′4′:6,7]indolizino[1,2-b]quinoline-3,14(4H,12H)-dione)) (“CPT”) and its derivatives are known as potent topoisomerase I inhibitors with broad-spectrum anticancer activities.

Topotecan (GlaxoSmithKline, U.S.A.) is a water-soluble, semisynthetic analogue of camptothecin, which has been approved by the U.S. Food and Drug Administration (FDA) for the treatment of epithelial ovarian cancer, cervical cancer and small cell lung cancer. The FDA recommended dose for recurrent ovarian cancer is of 1.5 mg/m² by 30-minute IV infusion for 5 consecutive days for a 21-day cycle. However, this daily Topotecan dose is associated with high incidence of myelosuppression and suboptimal patient convenience.

Hoskins et al. have investigated weekly topotecan in ovarian cancer patients and found that although weekly dosing arm was associated with less toxicity than the 5-day regime, the 5-day dosing regime was associated with a higher response rate than the weekly regime (Hoskins et al., Randomized phase II study of two schedules of topotecan in previously treated patients with ovarian cancer: a National Cancer Institute of Canada Clinical Trials Group study. Clin Oncol 1998; 16:2233-2237)

There is a need for an alternative dosing regimen of camptothecin and its derivative which is more convenient, less myelotoxic and clinically effective.

### BRIEF SUMMARY OF THE INVENTION

The present application provides for a method for inhibiting cancer cell growth in a subject, said method comprising the step of:

administering to said subject a composition comprising:

at least one compound of formula (I):

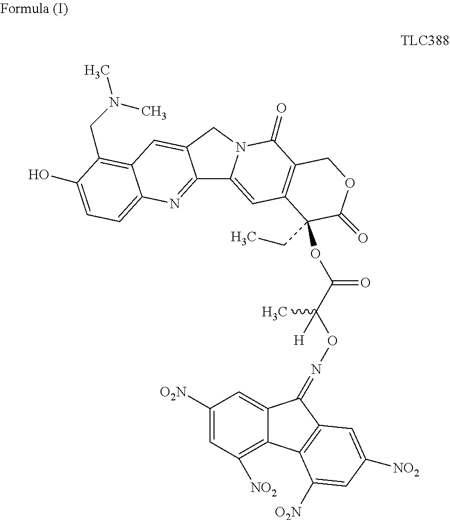 [SMILES: CC[C@@]1(OC(=O)C(C)ON=C2c3cc([N+](=O)[O-])cc([N+](=O)[O-])c3-c3c2cc([N+](=O)[O-])cc3[N+](=O)[O-])C(=O)OCc2c1cc1n(c2=O)Cc2cc3c(CN(C)C)c(O)ccc3nc2-1]

or a pharmaceutically acceptable salt thereof; and

at least one polyethylene glycol (PEG) conjugated phospholipid;

wherein the molar ratio of said PEG conjugated phospholipid to said compound or said pharmaceutically acceptable salt of said compound is greater than about 0.45:1;

wherein said composition provides a sustained release of a therapeutically effective amount of topotecan to said subject over a period of at least about 8 hours.

In one embodiment, methods of inhibiting cancer cell growth in a subject are provided, said method comprising administering to said subject as a composition comprising at least one compound of formula (I):

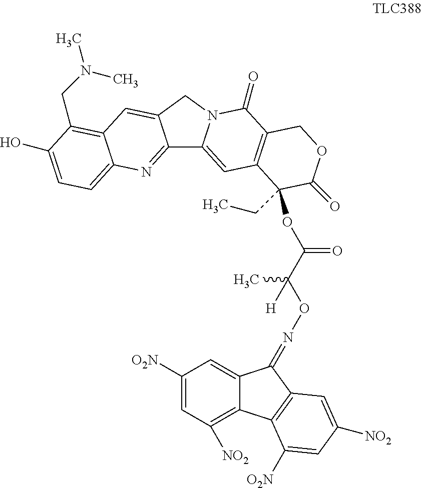 [SMILES: CC[C@@]1(OC(=O)C(C)ON=C2c3cc([N+](=O)[O-])cc([N+](=O)[O-])c3-c3c2cc([N+](=O)[O-])cc3[N+](=O)[O-])C(=O)OCc2c1cc1n(c2=O)Cc2cc3c(CN(C)C)c(O)ccc3nc2-1]

or a pharmaceutically acceptable salt thereof; and
at least one polyethylene glycol (PEG) conjugated phospholipid;
wherein the molar ratio of said PEG conjugated phospholipid to said compound or said pharmaceutically acceptable salt of said compound is greater than about 0.45:1; and
wherein said composition provides a sustained release of one or more of the following: topotecan, TLC-U1 and TLC-U2 to said subject over a period of at least about 1 hour.

### BRIEF DESCRIPTION OF THE DRAWINGS

FIG. 1 illustrates the postulated metabolic pathway of TLC388.

FIG. 2 illustrates the plasma disposition curves of TLC388HCl and its metabolites (Topotecan, TLC-U1 and TLC-U2) in subjects treated with 60 mg/m² of TLC388HCl (N=6).

FIG. 3 shows the type of cancer, doses of TLC388HCl composition and duration of treatment in patients with stable disease.

FIG. 4 is the CT images of a 70-year-old male with thymoma before and after 18 cycles of TLC388HCL treatment.

FIG. 5 illustrates the plasma disposition curves of TLC388HCl and its metabolites (Topotecan, TLC-U1 and TLC-U2) in subjects treated with 1.5 mg/m² of TLC388HCl (N=6).

### DETAILED DESCRIPTION OF THE INVENTION

### Definitions

As employed above and throughout the disclosure, the following terms, unless otherwise indicated, shall be understood to have the following meanings

As used herein, the singular forms “a,” “an,” and “the” include the plural reference unless the context clearly indicates otherwise.

A “single dose” as used herein, includes an intravenous infusion of 30 minutes duration or longer, or an oral administration of the composition.

The term “treating,” “treated,” or “treatment” as used herein includes preventative (e.g. prophylactic), palliative, and curative uses or results.

The term “inhibiting” and “suppressing” includes slowing or stopping the growth of.

The term “subject” includes a vertebrate having cancer. Preferably, the subject is a warm-blooded animal, including mammals, preferably humans.

As used herein, the term “administering” includes oral administration, administration as a suppository, topical contact, parenteral, intraperitoneal, intramuscular, intralesional, intranasal or subcutaneous administration, intrathecal administration, or the implantation of a slow-release device e.g., a mini-osmotic pump, to the subject.

“Pharmaceutical acceptable salt” includes acid addition salts. “Pharmaceutically acceptable acid addition salts” refer to those salts which retain the biological effectiveness and properties of the free bases, which are formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid and the like.

An “effective amount,” as used herein, includes an amount of the pharmaceutical composition or the present compound that is sufficient to reduce the symptoms and signs of cancer, such as mass, pain, and weight loss. As used herein, the term “therapeutically effective amount” is an amount sufficient to treat a specified disorder or inhibit cancer cells, or alternatively, to obtain a pharmacological response treating a disorder or inhibiting cancer cells.

“Micelles” are typically defined as spherical receptacles comprised of a single monolayer defining a closed compartment. Generally, amphipathic molecules such as surfactants and fatty acids spontaneously form micellar structures in polar solvents. Micelles typically have a spherical shape with the size of nanometer range. The formation of micelles is driven by decreasing free energy in the system because of removal of hydrophobic fragments from the aqueous environment and the re-establishment of hydrogen bond network with water molecules. In a micelle, there is an arrangement of polar amphipathic molecules, wherein the hydrophilic portion (i.e. heads) of the structure forms the exterior surface and the hydrophobic portion (i.e. tails) resides interiorly, away from the medium. Micelles do not have a bilayer structure and are not considered vesicles or liposomes. The compounds of the invention, when associated with micelles, are either in the compartment, bound to the micelles membrane, or bound to the outside surface of the micelle.

### The Pharmaceutical Compositions

Exemplary compositions that may be employed when implementing embodiments of the present invention are described in more detail in previously incorporated International Patent Application No. PCT/US2012/63447.

In one embodiment, the pharmaceutical composition comprises a compound, including, but not limited to, camptothecin, or at least one camptothecin derivative, salt, analogue, metabolite or pro-drug, or a pharmaceutically acceptable salt thereof; and at least one polyethylene glycol (PEG) conjugated phospholipid; wherein the molar ratio of said PEG conjugated phospholipid to said compound or said pharmaceutically acceptable salt of said compound is greater than about 0.45:1.

In one embodiment, the compound in the pharmaceutical composition comprises at least one compound of Formula (I):

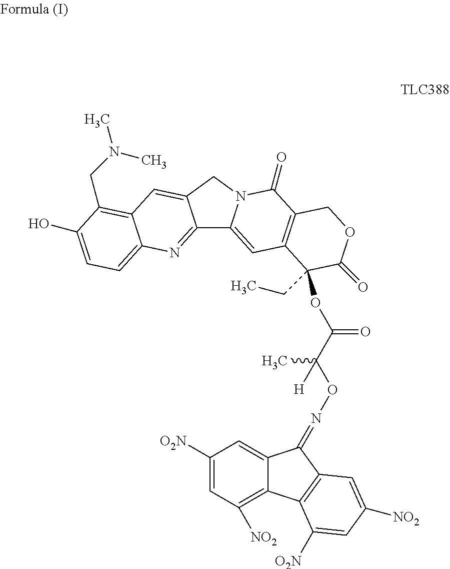 [SMILES: CC[C@@]1(OC(=O)C(C)ON=C2c3cc([N+](=O)[O-])cc([N+](=O)[O-])c3-c3c2cc([N+](=O)[O-])cc3[N+](=O)[O-])C(=O)OCc2c1cc1n(c2=O)Cc2cc3c(CN(C)C)c(O)ccc3nc2-1]

In another embodiment, the compound in the pharmaceutical composition comprises TLC388HCl with the following formulae:

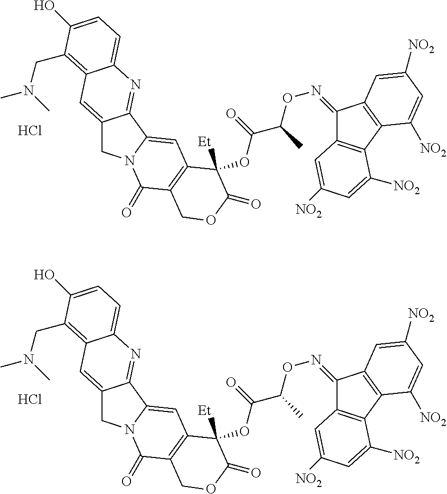 [SMILES: CC[C@@]1(OC(=O)[C@H](C)ON=C2c3cc([N+](=O)[O-])cc([N+](=O)[O-])c3-c3c2cc([N+](=O)[O-])cc3[N+](=O)[O-])C(=O)OCc2c1cc1n(c2=O)Cc2cc3c(CN(C)C)c(O)ccc3nc2-1 ; Cl ; CC[C@@]1(OC(=O)[C@@H](C)ON=C2c3cc([N+](=O)[O-])cc([N+](=O)[O-])c3-c3c2cc([N+](=O)[O-])cc3[N+](=O)[O-])C(=O)OCc2c1cc1n(c2=O)Cc2cc3c(CN(C)C)c(O)ccc3nc2-1 ; Cl]

TLC388HCl is a novel camptothecin analog with unique lactone ring modification, such modification result in improved stability, potency and lower incidence of side effect. It is a diastereomer and comprises (S,S) and (S,R) isomers in approximately 2:1 molar ratio. As used herein, the term “S” or “R” is a way to name an optical isomer by its configuration, without involving a reference molecule, which is called the R/S system. It labels each chiral center R or S according to a system by which its ligands are each assigned a priority, according to the Cahn Ingold Prelog priority rules, based on atomic number. This system labels each chiral center in a molecule (and also has an extension to chiral molecules not involving chiral centers). If the compound has two chiral centers, it can be labeled, for example, as an (S,S) isomer versus an (S,R) isomer.

The PEG moiety has a molecular weight from about 1,000 to about 20,000 daltons and is conjugated to the phospholipid moiety. The PEG conjugated phospholipid is mixed with the compound in the pharmaceutical composition, e.g., a compound of formula (I) or a pharmaceutically acceptable salt thereof, at a molar ratio of more than about 0.45:1. In one embodiment, the PEG conjugated phospholipid and the compound form micelles of substantially uniform size and narrow size distribution, which offer extended storage stability, improved solubility and reduced side effects.

The PEG conjugated phospholipid may be a PEG-DSPE (distearoyl-phosphatidylethanol amine) conjugate. In one embodiment, the PEG-DSPE conjugate is a methoxyl PEG-DSPE conjugate.

The molar ratio of the phospholipid to the compound in the pharmaceutical composition plays an important role in improving the stability of the compound. In a preferred embodiment, the PEG conjugated phospholipid is mixed with the compound at a molar ratio (lipid:compound) more than about 0.45:1. In a more preferred embodiment, the molar ratio of the phospholipid to the compound is from about 0.60:1 to about 1.00:1, and even more preferably, from about 0.70:1 to about 0.90:1. By mixing the phospholipid with the compound at the molar ratio as described herein, the micelles thus formed have an average diameter below about 40 nm, more particularly below about 20 nm, and even more particularly about 15 nm.

### pH Adjusting Agent

Certain CPT derivatives of the present invention, such as TLC388HCl, may be unstable in an alkaline environment. In a preferred embodiment, the pharmaceutical composition of the present invention has a pH less than about 4.0. In a more preferred embodiment, the pH of the pharmaceutical composition is between about 3 to about 4. The pharmaceutical composition may contain one or more pH adjusting agents to maintain an acidic pH and stabilizing the CPT derivatives. The pH adjusting agent can be any pharmaceutical acceptable buffer, which includes one or more of the following: oxalic acid, ethylenediamine tetraacetic acid, maleic acid, aspartic acid, phosphate, asparagine buffer, glycine, pyruvic acid, pyrophosphate, malonic acid, phthalate, fumaric acid, tartaric acid, citrate, furancarboxylic acid, β-alanine buffer, β:β′-dimethyl glutaric acid, formic acid, lactic acid, γ-aminobutyric acid, barbituric acid, benzoic acid, succinic acid, E-aminocaproic acid, acetic acid, propionic acid, malic acid, pyridine, histidine, cacodylic acid, carbonic acid, hydroxyimidazole, glycerol phosphate, ethylenediamine, imidazole, arsenic acid, 2,4,6-collidine, 1-, 2-, or 4-methyl imidazole, N-ethyl morpholine, veronal, barbital, 2,4-dimethyl imidazole, morpholine, N-ethyl morpholine, 2-amino-2-methyl-1,3-propanediol, 2-amino-2-ethyl-1,3-propanediol, diethanolamine, 4-aminopyridine, serine, boric acid, ammonia, ethanolamine, ephedrine, hydroxyproline, 2-amino-2-methyl-1-propanol, leucine, trimethyl, α-alanine, n-propyl alcohol, methylamine, ethylamine, n-butylamine, triethylamine, dimethylamine, hexamethylenediamine, piperidine, p-toluenesulfonic acid, tris(hydroxymethyl)aminomethane (Tris), glycylglycine, GTA buffer, Good buffer such as MES buffer, Bis-Tris buffer, ADA buffer, PIPES buffer, ACES buffer, MOPSO buffer, BES buffer, MOPS buffer, TES buffer, HEPES buffer, DIPSO buffer, TAPSO buffer, POPSO buffer, HEPPSO buffer, EPPS buffer, Tricine buffer, Bicine buffer, TAPS buffer, CHES buffer, CAPSO buffer, and CAPS buffer. Preferably, the pH adjusting agent comprises one or more of the following: citrate, fumaric acid, diethanolamine, Tris, glycine, acetic acid, succinic acid, tartaric acid, carbonic acid, imidazole and maleic acid.

The pharmaceutical composition of the invention may further comprise at least one cryoprotectant such as mannitol, glycerol, dextrose, sucrose, and/or trehalose. One preferred cryoprotectant is mannitol.

In some embodiments, this invention also provides a pharmaceutical composition further comprising at least one pharmaceutically acceptable excipient, diluent, vehicle, medium for the active ingredient, or a combination.

In one embodiment, the pharmaceutical composition comprising TLC388HCl or the pharmaceutically acceptable salt of TLC388 HCL; methoxyl PEG-DSPE (distearoyl-phosphatidylethanolamine) conjugate; and citric acid, wherein the methoxyl PEG conjugated phospholipid is mixed with the TLC388HCl or the pharmaceutically acceptable salt of TLC388 HCL at a molar ratio of between about 0.45:1 to about 0.9:1.

The pharmaceutical compositions may be in a form suitable for oral use, for example, as tablets, troches, lozenges, aqueous or oily suspensions, dispersible powders or granules, emulsions and self emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

The pharmaceutical compositions may be in the form of a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In one embodiment, methods for inhibiting cancer cell growth in a subject, including administering to the subject an effective amount of a pharmaceutical composition of the invention are provided.

The dosage of the sustained release composition of the present invention can be determined by the skilled person in the art according to the embodiments. A single dose or multiple dose forms are contemplated, each offering advantages in certain clinical settings. According to the present invention, the actual amount of the pharmaceutical composition to be administered can vary in accordance with the age, weight, condition of the subject to be treated, the type of cancer, the incidence of side effect, and depends on the discretion of medical professionals.

In one embodiment, for the purpose of inhibiting cancer cells, the pharmaceutical composition of the present invention can be administered such that the dosage of the compound of formula (I) or TLC388HCl ranges from about 1.0 mg/m²/dose to about 70 mg/m²/dose. In another embodiment, a dosage of the compound of formula (I) or TLC388HCl ranges from about 1.5 mg/m²/dose to about 60 mg/m²/dose or any dose or ranges of dose therebetween in 0.5 mg/m² increments (e.g., about 9 mg/m², about 13.5 mg/m², about 35 to about 50 mg/m², etc.).

The pharmaceutical composition of the present invention can be administered as a substantially single dose or can be repeated at a weekly interval. In one embodiment, the pharmaceutical composition is administered as 3 weekly IV bolus infusions over a 28-day cycle for inhibiting cancer cell growth. In another embodiment, the pharmaceutical composition is administered on day 1, day 8 and day 15 of a 28-day cycle for inhibiting cancer cell growth.

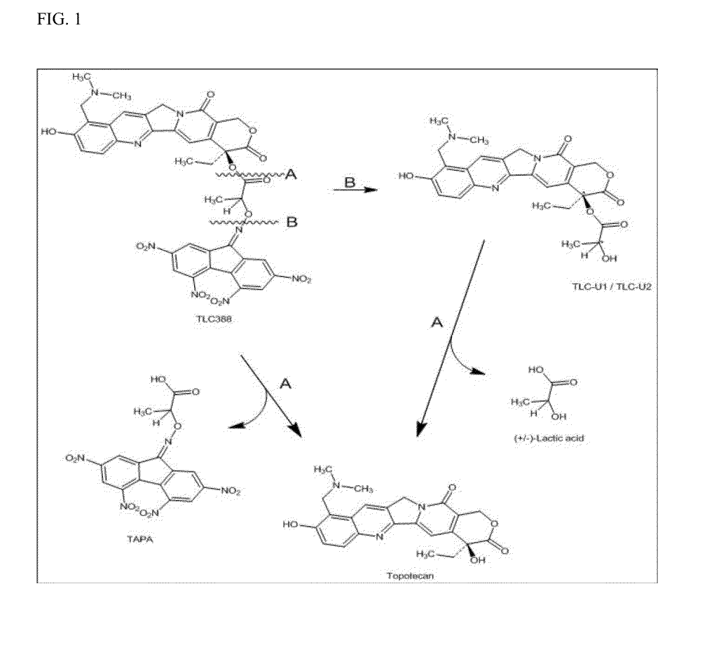

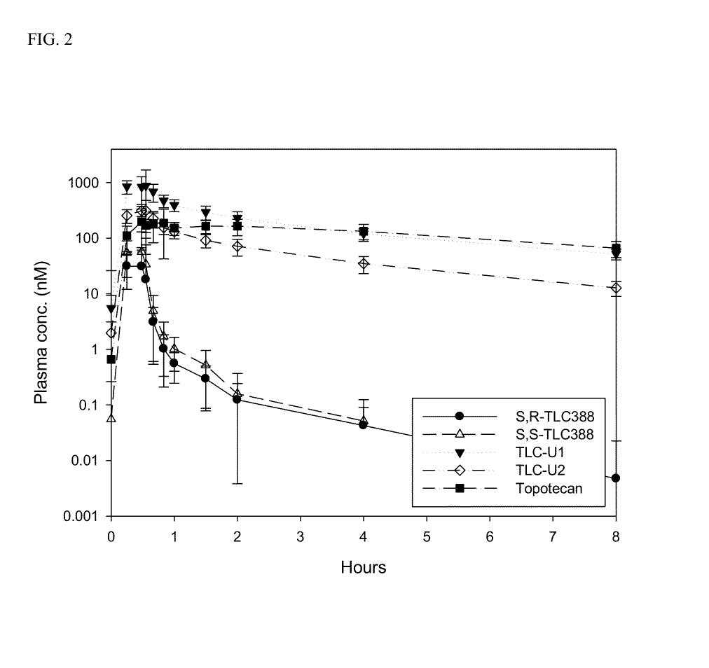

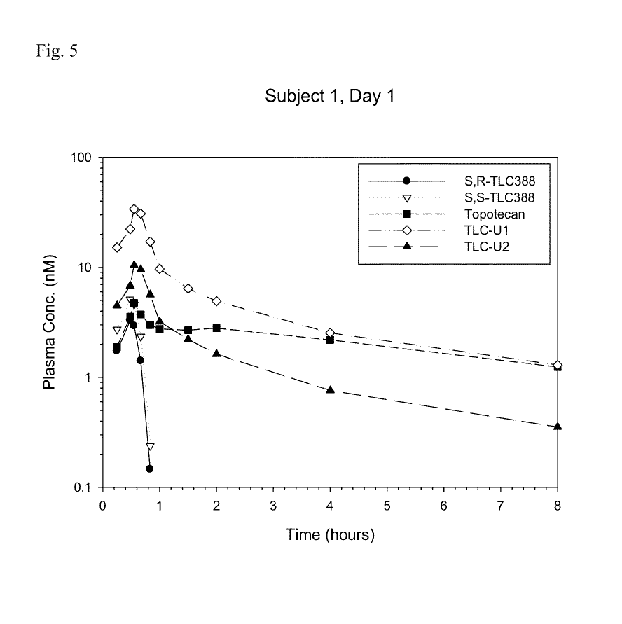

Without being bound by any particular theory, it is believed that TLC388 is metabolized to topotecan, TLC-U1 and TLC-U2 in vivo (see TLC388 metabolic pathway in FIG. 1). The pharmaceutical composition of the present invention provides a sustained release of one or more of the following metabolites: topotecan, TLC-U1 and TLC-U2, as active ingredients for at least 1 hour (see FIG. 2 and FIG. 5).

TLC-U1 is S,R-topotecan lactate, with the following formula:

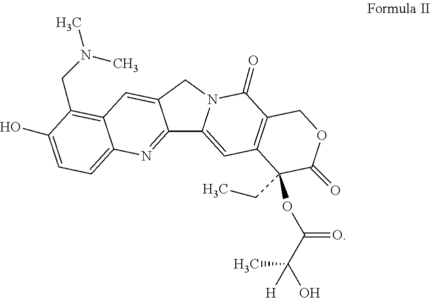 [SMILES: CC[C@@]1(OC(=O)[C@@H](C)O)C(=O)OCc2c1cc1n(c2=O)Cc2cc3c(CN(C)C)c(O)ccc3nc2-1]

TLC-U2 is S,S-topotecan lactate with the following formula:

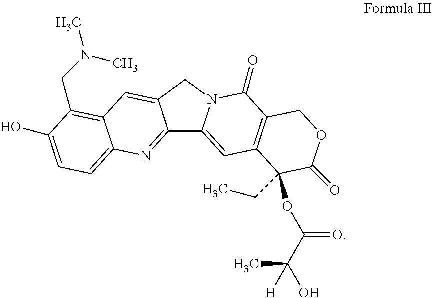 [SMILES: CC[C@@]1(OC(=O)[C@H](C)O)C(=O)OCc2c1cc1n(c2=O)Cc2cc3c(CN(C)C)c(O)ccc3nc2-1]

Both TLC-U1 and TLC-U2 have been disclosed in U.S. Pat. No. 8,168,648, which is incorporated by reference in its entirety.

TLC-U1 and TLC-U2 are weak topoisomerase I Inhibitors. However, in-vitro study shows the anti-proliferative activity of TLC-U2 was comparable to that of TLC 388HCl, and more potent than that of TLC-U1, suggesting its anti-proliferative activity is unrelated to topoisomerase I inhibition.

As used herein, the term “sustained release” refers to the continuous release of one or more compound or metabolite over a predetermined time period and at a level sufficient to achieve a desired effect throughout the predetermined time period. In some embodiments, a composition described herein provides a sustained release of topotecan, TLC-U1 and/or TLC-U2 over a period of at least about 1 hour, at least about 2 hours, at least about 3 hours, at least about 4 hours, at least about 5 hours, at least about 6 hours, at least about 7 hours, at least about 8 hours, ranging from about 2 hours to about 8 hours, ranging from about 4 hours to about 8 hours, ranging from about 6 hours to about 8 hours, at least about 8 hours or more, ranging from about 8 hours to about 12 hours, ranging from about 12 hours to about 24 hours, ranging from about 24 hours to about 48 hours, ranging from about 48 hours to about 72 hours, ranging from about 1 week to about 3 weeks, from about 3 weeks to about 6 weeks.

In one embodiment, the present methods provides a sustained release of topotecan and/or TLC-U1 at a level of greater than about 0.3 nM, about 1 nM, about 2 nM, about 5 nM, about 10 nM, about 40 nM 1 hour post drug administration, or from about 1 nM to about 40 nM, from about 2 nM to about 30 nM, from about 5 to about 10 nM 1 hour after the administration of the composition.

In the present methods, the composition provides a sustained release of a therapeutically effective amount of topotecan, TLC-U1, and/or TLC-U2 over a time period. In one embodiment, the Cₘₐₓ (peak plasma concentration) of topotecan may range from about 0.1 ng/mL to about 4000 ng/mL, from about 1 ng/mL to about 3720 ng/mL, or from about 25 ng/mL to about 3720 ng/mL. In a second embodiment, the tₘₐₓ (time to reach the peak plasma concentration) of topotecan may range from about 0.1 to about 2.5 hours, from about 0.4 hours to about 1.9 hours, or from about 0.8 hours to about 1.0 hours. In a third embodiment, the AUC₀₋₈ₕᵣ (area under the curve) of topotecan may range from about 1 hr·ng/mL to about 550 hr·ng/mL, from about 5 hr·ng/mL to about 500 hr·ng/mL, or from about 140 hr·ng/mL to about 500 hr·ng/mL. In a fourth embodiment, the t_{1/2} (half-life) of topotecan may range from about 2 hours to about 15 hours, from about 4 hours to about 12.5 hours, or from about 4.5 hours to about 7.5 hours.

In one embodiment, the Cₘₐₓ of TLC-U2 may range from about 1 ng/mL to about 150 ng/mL, from about 2 ng/mL to about 105 ng/mL, or from about 50 ng/mL to about 110 ng/mL. In another embodiment, the tₘₐₓ of TLC-U2 may range from about 0.1 hour to about 1 hour, from about 0.2 hours to about 0.7 hours, or from about 0.3 hours to about 0.5 hours. In yet another embodiment, the AUC₀₋₈ₕᵣ of TLC-U2 may range from about 1 hr·ng/mL to about 130 hr·ng/mL, from about 2.5 hr·ng/mL to about 115 hr·ng/mL or from about 70 hr·ng/mL to about 115 hr·ng/mL. In yet another embodiment, the t_{1/2} of TLC-U2 may range from about 1 hour to about 5 hours, from about 1.5 hours to about 3.5 hours, or from about 1.5 hours to about 3.0 hours.

In one embodiment, the Cₘₐₓ of TLC-U1 may range from about 1 ng/mL to about 500 ng/mL, from about 5 ng/mL to about 450 ng/mL, or from about 200 ng/mL to about 450 ng/mL. In another embodiment, the tₘₐₓ of TLC-U1 may range from about 0.1 hour to about 1 hour, from about 0.2 hours to about 0.7 hours, or from about 0.3 hours to about 0.6 hours. In yet another embodiment, the AUC₀₋₈ₕᵣ of TLC-U1 may range from about 1 hr·ng/mL to about 500 hr·ng/mL, from about 5 hr·ng/mL to about 430 hr·ng/mL or from about 270 hr·ng/mL to about 430 hr·ng/mL. In yet another embodiment, the t_{1/2} of TLC-U1 may range from about 1 hour to about 7 hours, from about 1.5 hours to about 5.6 hours, or from about 1.8 hours to about 3.5 hours.

The following examples further illustrate the present invention. These examples are intended merely to be illustrative of the present invention and are not to be construed as being limiting.

### Example 1

### Weekly Injection of Pharmaceutical Composition in Cancer Cell Inhibition

A non-randomized phase I clinical trial was conducted at 4 institutions in the US and Taiwan.

Patients were included in this trial if they are over the age of 18, with advanced cancer and the ECOG performance status is 0 or 1. (For ECOG performance status, see Oken et al. Toxicity and Response Criteria of the Eastern Cooperative Oncology Group. Am J Clin Oncol 5:649-655, 1982). Patients were excluded from the study if they have previously received more than 3 regimens of chemotherapy, or if they have received chemotherapy or radiotherapy within 4 weeks of the trial recruitment.

Fifty eight patients were enrolled in the trial, of which 54 patients received the pharmaceutical composition of the present invention. Table 1 shows the demographic data of the enrolled patients.

TABLE 1
Patient Demographic and Baseline characteristics

| Total patients treated | N = 54 | Percentage |
|---|---|---|
| Age, years | | |
| Mean (SD) | 60.5 (11.57) | |
| Median | 62.0 | |
| Range | 33-80 | |
| Sex | | |
| Female | 22 | 41 |
| Male | 32 | 59 |
| Race | | |
| Caucasian | 18 | 33 |
| Black | 16 | 30 |
| Asian | 15 | 28 |
| Hispanic | 5 | 9 |
| ECOG status | | |
| 0 | 12 | 22 |
| 1 | 42 | 78 |
| Primary cancer diagnosis | | |
| Prostate | 9 | 17 |
| Colon | 8 | 15 |
| Esophagus | 4 | 7 |
| Pancreas | 4 | 7 |
| Cervix | 3 | 6 |
| Gallbladder | 3 | 6 |
| Kidney | 3 | 6 |
| Others | 20 | 37 |
| No. of metastatic disease sites | | |
| 0 site | 1 | 2 |
| 1 site | 35 | 64 |
| 2 sites | 7 | 13 |
| >3 sites | 11 | 20 |
| Metastatic disease site | | |
| Liver | 21 | 39 |
| Lung | 20 | 37 |
| Lymph node | 11 | 20 |
| Bone | 8 | 15 |
| Others | 15 | 28 |
| Types of prior cancer therapies | | |
| Chemo + radiation + surgery | 19 | 35 |
| Chemo + surgery | 23 | 43 |
| Radiation + chemo | 4 | 7 |
| Cytokine + surgery | 1 | 2 |
| Chemo only | 4 | 7 |
| Cytokine only | 1 | 2 |
| Surgery only | 2 | 4 |
| No. of prior chemotherapy regimens | | |
| 0 | 4 | 7 |
| 1 | 7 | 13 |
| 2 | 19 | 35 |
| 3 | 24 | 44 |

Eligible patients were given the pharmaceutical composition of the present invention, comprising a mixture of TLC388HCl and mPEG2000-DSPE in a molar ratio of 1:0.7 to 1:0.9 (known as Lipotecan®). 1 Gm of TLC388HCl contains 260 mg of TLC388 base. The pharmaceutical composition was administered as a 30-min IV infusion, on days 1, 8, and 15 of a 28-day cycle (i.e. 3 IV infusions per 28-day cycle).

A “3+3” standard dose-escalation design was used to establish the Maximum Tolerated Dose of TLC388HCl composition. The dose could be increased or escalated in subsequent cycle(s) if there is no dose limiting toxicity (DLT) event in a cohort of 3 patients (0/3) or 1 DLT event in two cohorts (6 patients) in the previous cycle, and the does is reduced if 2 DLT events are observed in two cohorts (6 patients) in the previous cycle. The dose was adjusted by the Safety Review Committee. The dose escalation is summarized in Table 2.

TABLE 2
Dose Escalation Summary

| | | | Number of Patient | | | |
|---|---|---|---|---|---|---|
| | | | Subsequently Dose | | # of | # of Patient |
| Co- | Dose | Patient | Esca- | Re- | Cycle | with DLT |
| hort | (mg/m²) | Number | lated | duced | Completed | @1^{st} Cycle |
| 1 | 1.5 | 3 | 0 | 0 | 5 | 0 |
| 2 | 3.0 | 4 | 2 | 0 | 14 | 0 |
| 3 | 6.0 | 3 | 1 | 0 | 9 | 0 |
| 4 | 9.0 | 4 | 0 | 0 | 8 | 0 |
| 5 | 13.5 | 4 | 0 | 0 | 6 | 0 |
| 6 | 20 | 4 | 0 | 0 | 6 | 0 |
| 7 | 30 | 4 | 0 | 0 | 5 | 0 |
| 8 | 40 | 6 | 1 | 0 | 41 | 2 |
| 9 | 35 | 4 | 2 | 0 | 19 | 0 |
| 10 | 40 | 7 | 3 | 1 | 31 | 1 |
| 11 | 50 | 5 | 0 | 1 | 7 | 1 |
| 12 | 60 | 6 | 0 | 1 | 14 | 2 |
| Total | | 54 | 9 | 3 | 165 | 6 |

Forty-five (83%) patients completed the 3 weekly infusions in Cycle 1, and the highest TLC388HCl dose was 60.0 mg/m².

Serum samples were collected from the patients for pharmacokinetic analysis on day 1, day 8 and day 15 of Cycles 1 and 2, at the following times:

During the 30-minute IV infusion: 0 (immediately prior to IV infusion); 15 min (Day 1 and 8 of Cycle 1 only); 29 min or as close to end of infusion as or possible, in any case before end of the 30 minute infusion.

Post IV infusion: (Day 1 and 8 of Cycle 1): 33 min; 40 min; 50 min; 1 h; 1 h 30 min; 2 h; 4 h and 8 h. Post IV infusion (Day 15 of Cycle 1 and Day 1, 8, and 15 of Cycle 2): 4 h. Patients were assessed every 8 weeks, after 2 cycles of treatments. Patients had the option to continue the TLC388HCl treatment if there was no disease progression and the side effects were tolerable.

During the 2-cycle study period, the patients were examined regularly for the following outcomes:
Side effect/DLT;
The pK profiles of the (S,S) and (S,R) diastereomers of TLC388HCl, its 3 metabolites (TLC-U1, TLC-U2 and Topotecan) on day 1 and day 8 of the treatment cycle; and
Anti-cancer efficacy.

### Results:

1. Summary of Side Effects:

Table 3 shows the common side effects reported by at least 10% of the patients during the trial. TLC388HCl composition of the present invention was generally well tolerated and the common side effects are similar to those seen in other topoisomerase inhibitors, such as bone marrow suppression (anemia, neutropenia and thrombocytopenia) and gastrointestinal symptoms such as nausea, vomiting, diarrhea and abdominal pain. The incidence of side effect was similar between the overall group and the groups receiving 2 highest doses (50 and 60 mg/m²), except for bone marrow suppression. The incidences of neutropenia (30% overall and 64% in the two highest dose groups), thrombocytopenia (28% overall and 45% in the two highest dose groups), and leukopenia (26% overall and 45% in the two highest dose groups) were much higher in the two highest dose groups. Tables 4 and 5 further illustrate the incidences of various side effects in the higher dose groups (40 to 60 mg/m²).

TABLE 3
Common Side Effects Reported By Patients

| | Number of patients (%) | | | |
|---|---|---|---|---|
| | Overall | | 50 and 60 mg/m² | |
| | N = 54 | | N = 11 | |
| Adverse event | Number of patient (%) | | Number of patient (%) | |
| Anaemia | 36 | (67) | 7 | (64) |
| Fatigue | 22 | (41) | 6 | (55) |
| Nausea | 20 | (37) | 5 | (45) |
| Neutropenia | 16 | (30) | 7 | (64) |
| Thrombocytopenia | 15 | (28) | 5 | (45) |
| Diarrhea | 15 | (28) | 2 | (18) |
| Leukopenia | 14 | (26) | 5 | (45) |
| Constipation | 14 | (26) | 4 | (36) |
| Vomiting | 14 | (26) | 4 | (36) |
| Decreased appetite | 12 | (22) | 1 | (9) |
| Abdominal pain | 11 | (20) | 1 | (9) |
| Peripheral Oedema | 9 | (17) | 2 | (18) |
| Dizziness | 8 | (15) | 0 | |
| Cough | 7 | (13) | 0 | |
| Hypoalbuminaemia | 7 | (13) | 2 | (18) |
| Hypocalcaemia | 7 | (13) | 2 | (18) |
| Hyponatraemia | 7 | (13) | 0 | |
| Abdominal pain upper | 6 | (11) | 1 | (9) |
| Back pain | 6 | (11) | 1 | (9) |
| Urinary tract infection | 5 | (9) | 2 | (18) |

Table 4 shows the side effect possibly or probably related, or related to the higher dose (40 mg/m² to 60 mg/m²) of TLC388HCl Composition.

| | All patients | | 40 mg/m² | | 50 mg/m² | | 60 mg/m² | |
|---|---|---|---|---|---|---|---|---|
| | N = 54 | | N = 13 | | N = 5 | | N = 6 | |
| Adverse Event | No. | % | No. | % | No. | % | No. | % |
| Neutropenia | 14 | 26 | 5 | 38 | 3 | 60 | 3 | 50 |
| Anemia | 10 | 19 | 4 | 31 | 1 | 20 | 2 | 33 |
| Leukopenia | 8 | 15 | 2 | 15 | 2 | 40 | 2 | 33 |
| Thrombocytopenia | 8 | 15 | 3 | 23 | 1 | 20 | 2 | 33 |
| Hyponatremia | 2 | 4 | 2 | 15 | 0 | 0 | 0 | 0 |
| Diarrhea | 1 | 2 | 1 | 8 | 0 | 0 | 0 | 0 |
| Febrile neutropenia | 1 | 2 | 0 | 0 | 0 | 0 | 1 | 17 |
| Hypophosphatemia | 1 | 2 | 0 | 0 | 0 | 0 | 1 | 17 |
| Cerebrovascular | 1 | 2 | 0 | 0 | 0 | 0 | 0 | 0 |
| Accident | | | | | | | | |

Table 5 shows the DLT of the higher doses (40 mg/m² to 60 mg/m²) TLC388HCl

| Dose | Adverse Event | | NCI-CTCAE |
|---|---|---|---|
| (mg/m²) | Preferred Term | Relationship | Toxicity |
| 40.0 | Hyponatraemia^{1,2} | Possibly | Life-threatening |
| 40.0 | Thrombocytopenia^{1,3,4} | Probably | Life-threatening |
| 40.0 | Hyponatraemia² | Possibly | Severe |
| 50.0 | Thrombocytopenia³ | Definitely | Life-threatening |
| 60.0 | Neutropenia² | Definitely | Severe |
| 60.0 | Febrile neutropenia^{1,3} | Definitely | Severe |

¹Serious Adverse event
²Discontinued TLC388
³TLC388 temporarily witheld
⁴TLC388 dose reduced

The pK profiles of S,S TLC388HCl in Day 1 and Day 8 are shown in Table 6 and Table 7, respectively.

TABLE 6
Day 1 pK profiles of S,S TLC388HCl for various doses of TLC388HCl

| | Pharmacokinetic Parameter, Mean (SD) | | | | | |
|---|---|---|---|---|---|---|
| Dose Group | AUC₀₋ₜ | | Cₘₐₓ | | Tₘₐₓ | t_{1/2} |
| (mg/m²) | (hr · ng/mL) | | (ng/mL) | | (hr) | (hr) |
| 1.5 | 2.190 | (2.043) | 4.492 | (3.619) | 0.606 (0.106) | 2.792 (3.856) |
| 3.0 | 6.136 | (9.117) | 3.857 | (2.329) | 0.933 (0.936) | — |
| 6.0 | 1.866 | (1.782) | 5.763 | (3.853) | 0.467 (0.209) | — |
| 9.0 | 1.141 | (1.537) | 2.985 | (4.054) | 0.429 (0.212) | 0.074 (0.033) |
| 13.5 | 9.096 | (9.946) | 10.223 | (9.334) | 0.438 (0.375) | 0.449 (0.525) |
| 20.0 | 14.699 | (16.49) | 51.193 | (64.56) | 0.250 (0.000) | 0.432 (0.260) |
| 30.0 | 5.227 | (3.849) | 18.623 | (18.53) | 0.454 (0.276) | 0.160 (0.034) |
| 35.0 | 212.19 | (229.6) | 417.03 | (535.7) | 0.679 (0.116) | 2.221 (0.789) |
| 40.0 | 17.287 | (23.77) | 47.220 | (57.64) | 0.415 (0.152) | 0.457 (0.812) |
| 50.0 | 23.967 | (12.87) | 76.840 | (56.09) | 0.403 (0.143) | 0.218 (0.075) |
| 60.0 | 18.196 | (22.50) | 103.16 | (176.0) | 0.357 (0.148) | 0.546 (0.353) |

TABLE 7
Day 8 pK profiles of S,S TLC388HCl for various doses of TLC388HCl

| | Pharmacokinetic Parameter, Mean (SD) | | | | | |
|---|---|---|---|---|---|---|
| Dose Group | AUC₀₋ₜ | | Cₘₐₓ | | Tₘₐₓ | t_{1/2} |
| (mg/m²) | (hr · ng/mL) | | (ng/mL) | | (hr) | (hr) |
| 1.5 | 0.608 | (0.686) | 1.297 | (1.320) | 0.489 (0.215) | 0.093 |
| 3.0 | 0.852 | (0.927) | 2.902 | (3.430) | 0.350 (0.173) | — |
| 6.0 | 3.152 | (1.415) | 12.160 | (7.606) | 0.467 (0.209) | 0.045 |
| 9.0 | 7.000 | (9.669) | 4.776 | (5.282) | 2.408 (3.729) | 0.195 |
| 13.5 | 4.343 | (2.976) | 9.020 | (6.767) | 0.263 (0.225) | 1.580 (0.305) |
| 20.0 | 7.369 | (3.959) | 24.533 | (15.56) | 0.328 (0.135) | 0.509 (0.731) |
| 30.0 | 28.454 | (47.44) | 58.903 | (92.21) | 0.587 (0.167) | 0.114 (0.061) |
| 35.0 | 208.58 | (285.2) | 616.50 | (537.5) | 0.621 (0.141) | 4.009 (1.684) |
| 40.0 | 58.170 | (101.2) | 127.39 | (204.0) | 0.303 (0.113) | 0.429 (0.687) |
| 50.0 | 58.594 | (94.95) | 150.12 | (214.6) | 0.297 (0.104) | 0.817 (1.200) |
| 60.0 | 24.500 | (15.91) | 87.780 | (67.19) | 0.403 (0.143) | 0.971 (0.667) |

The pK profiles of S,R TLC388HCl in Day 1 and Day 8 are shown in Table 8 and Table 9, respectively.

TABLE 8
Day 1 pK profiles of S,R TLC388HCl for various doses of TLC388HCl

| | Pharmacokinetic Parameter, Mean (SD) | | | | | |
|---|---|---|---|---|---|---|
| Dose Group | AUC₀₋ₜ | | Cₘₐₓ | | Tₘₐₓ | t_{1/2} |
| (mg/m²) | (hr · ng/mL) | | (ng/mL) | | (hr) | (hr) |
| 1.5 | 1.646 | (1.637) | 3.188 | (2.709) | 0.606 (0.106) | 2.603 (3.591) |
| 3.0 | 4.728 | (7.172) | 2.743 | (1.593) | 0.933 (0.936) | — |
| 6.0 | 1.564 | (1.184) | 4.073 | (2.952) | 0.467 (0.209) | — |
| 9.0 | 0.821 | (1.175) | 2.196 | (3.124) | 0.354 (0.209) | 0.197 (0.218) |
| 13.5 | 5.922 | (6.548) | 6.448 | (5.735) | 0.438 (0.375) | 0.485 (0.575) |
| 20.0 | 8.671 | (8.969) | 29.303 | (35.87) | 0.250 (0.000) | 2.828 (4.510) |
| 30.0 | 3.624 | (2.941) | 12.930 | (13.77) | 0.396 (0.292) | 0.258 (0.271) |
| 35.0 | 138.61 | (149.4) | 263.58 | (336.4) | 0.709 (0.083) | 3.030 (1.537) |
| 40.0 | 10.818 | (13.86) | 28.259 | (30.66) | 0.415 (0.152) | 0.460 (0.772) |
| 50.0 | 14.387 | (6.216) | 43.180 | (23.76) | 0.403 (0.143) | 0.483 (0.334) |
| 60.0 | 11.285 | (13.71) | 63.194 | (107.0) | 0.357 (0.148) | 0.639 (0.425) |

TABLE 9
Day 8 pK profiles of S,R TLC388HCl for various doses of TLC388HCl

| | Pharmacokinetic Parameter, Mean (SD) | | | | | |
|---|---|---|---|---|---|---|
| Dose Group | AUC₀₋ₜ | | Cₘₐₓ | | Tₘₐₓ | t_{1/2} |
| (mg/m²) | (hr · ng/mL) | | (ng/mL) | | (hr) | (hr) |
| 1.5 | 0.449 | (0.510) | 0.916 | (0.963) | 0.489 (0.215) | 0.087 |
| 3.0 | 0.574 | (0.557) | 1.811 | (1.875) | 0.350 (0.173) | 0.050 (0.008) |
| 6.0 | 2.190 | (1.185) | 8.287 | (5.455) | 0.467 (0.209) | 0.072 |
| 9.0 | 5.396 | (7.801) | 3.325 | (3.783) | 2.408 (3.729) | 0.133 (0.084) |
| 13.5 | 3.047 | (1.896) | 5.948 | (4.200) | 0.263 (0.225) | 0.811 (0.907) |
| 20.0 | 4.707 | (2.763) | 15.753 | (9.943) | 0.328 (0.135) | 0.115 (0.064) |
| 30.0 | 18.892 | (32.28) | 38.413 | (63.12) | 0.512 (0.240) | 0.202 (0.242) |
| 35.0 | 139.26 | (187.7) | 403.60 | (355.5) | 0.621 (0.141) | 3.329 (2.590) |
| 40.0 | 34.254 | (57.95) | 76.660 | (122.0) | 0.303 (0.113) | 0.411 (0.676) |
| 50.0 | 39.251 | (64.55) | 97.288 | (140.3) | 0.297 (0.104) | 0.764 (1.045) |
| 60.0 | 13.377 | 8.345 | 47.340 | (33.52) | 0.450 (0.115) | 1.416 (1.169) |

The pK profiles of TLC-U1 in Day 1 and Day 8 are shown in Table 10 and Table 11, respectively.

TABLE 10
Day 1 pK profiles of TLC-U1 for various doses of TLC388HCl

| | Pharmacokinetic Parameter, Mean (SD) | | | |
|---|---|---|---|---|
| Dose Group | AUC₀₋ₜ | Cₘₐₓ | Tₘₐₓ | t_{1/2} |
| (mg/m²) | (hr · ng/mL) | (ng/mL) | (hr) | (hr) |
| 1.5 | 13.013 (0.770) | 11.207 (1.374) | 0.628 (0.068) | 2.792 (0.343) |
| 3.0 | 24.583 (9.544) | 17.133 (0.902) | 0.467 (0.209) | 2.652 (0.943) |
| 6.0 | 44.302 (5.850) | 28.467 (1.185) | 0.467 (0.209) | 2.896 (0.352) |
| 9.0 | 48.590 (22.11) | 37.775 (17.95) | 0.563 (0.076) | 3.677 (0.919) |
| 13.5 | 99.094 (56.53) | 56.275 (3.500) | 0.308 (0.117) | 2.849 (0.533) |
| 20.0 | 111.91 (46.50) | 87.400 (51.76) | 0.405 (0.135) | 2.950 (0.479) |
| 30.0 | 162.64 (18.15) | 104.90 (13.24) | 0.454 (0.276) | 2.661 (0.251) |
| 35.0 | 339.60 (33.05) | 195.50 (25.27) | 0.638 (0.059) | 5.329 (3.327) |
| 40.0 | 282.81 (66.21) | 202.40 (74.63) | 0.392 (0.159) | 2.943 (0.833) |
| 50.0 | 365.36 (106.4) | 242.40 (59.20) | 0.436 (0.104) | 2.900 (0.870) |
| 60.0 | 420.79 (89.67) | 346.60 (242.7) | 0.357 (0.148) | 2.908 (0.539) |

TABLE 11
Day 8 pK profiles of TLC-U1 for various doses of TLC388HCl

| | Pharmacokinetic Parameter, Mean (SD) | | | |
|---|---|---|---|---|
| Dose Group | AUC₀₋ₜ | Cₘₐₓ | Tₘₐₓ | t_{1/2} |
| (mg/m²) | (hr · ng/mL) | (ng/mL) | (hr) | (hr) |
| 1.5 | 10.869 (1.365) | 6.897 (1.325) | 0.483 (0.000) | 2.687 (0.329) |
| 3.0 | 16.705 (4.893) | 13.900 (5.956) | 0.467 (0.209) | 3.059 (1.788) |
| 6.0 | 46.533 (10.74) | 42.600 (21.84) | 0.483 (0.000) | 2.716 (0.092) |
| 9.0 | 54.481 (10.70) | 30.550 (13.03) | 0.529 (0.240) | 3.319 (0.665) |
| 13.5 | 68.322 (24.11) | 60.800 (12.91) | 0.263 (0.225) | 1.867 (1.046) |
| 20.0 | 101.46 (37.00) | 84.400 (28.61) | 0.405 (0.135) | 2.385 (0.217) |
| 30.0 | 190.59 (33.45) | 132.00 (34.24) | 0.512 (0.240) | 2.750 (0.317) |
| 35.0 | 406.54 (77.86) | 327.75 (216.8) | 0.546 (0.087) | 4.064 (1.658) |
| 40.0 | 285.94 (207.9) | 435.70 (848.7) | 0.410 (0.180) | 2.163 (0.707) |
| 50.0 | 349.27 (131.8) | 240.20 (115.7) | 0.417 (0.155) | 3.289 (0.640) |
| 60.0 | 417.98 (54.75) | 308.00 (74.92) | 0.496 (0.030) | 2.808 (0.507) |

The pK profiles of TLC-U2 in Day 1 and Day 8 are shown in Table 12 and Table 13, respectively.

TABLE 12
Day 1 pK profiles of TLC-U2 for various doses of TLC388HCl

| | Pharmacokinetic Parameter, Mean (SD) | | | |
|---|---|---|---|---|
| Dose Group | AUC₀₋ₜ | Cₘₐₓ | Tₘₐₓ | t_{1/2} |
| (mg/m²) | (hr · ng/mL) | (ng/mL) | (hr) | (hr) |
| 1.5 | 3.736 (0.700) | 3.343 (0.499) | 0.628 (0.068) | 2.307 (0.535) |
| 3.0 | 7.908 (3.610) | 5.137 (0.899) | 0.467 (0.209) | 2.337 (0.706) |
| 6.0 | 11.576 (1.629) | 7.517 (0.400) | 0.467 (0.209) | 2.463 (0.256) |
| 9.0 | 15.465 (6.671) | 11.530 (4.799) | 0.504 (0.178) | 3.258 (0.959) |
| 13.5 | 29.054 (15.78) | 18.075 (2.685) | 0.367 (0.135) | 2.449 (0.178) |
| 20.0 | 33.636 (11.27) | 28.600 (15.93) | 0.483 (0.000) | 2.480 (0.537) |
| 30.0 | 49.544 (10.29) | 31.950 (2.922) | 0.425 (0.117) | 2.323 (0.304) |
| 35.0 | 88.171 (13.21) | 48.475 (7.776) | 0.609 (0.068) | 3.126 (0.635) |
| 40.0 | 74.133 (21.71) | 52.460 (17.84) | 0.445 (0.146) | 2.622 (0.830) |
| 50.0 | 100.79 (32.17) | 65.660 (17.76) | 0.390 (0.128) | 2.548 (0.680) |
| 60.0 | 106.95 (23.66) | 87.400 (49.49) | 0.450 (0.115) | 2.552 (0.422) |

TABLE 13
Day 8 pK profiles of TLC-U2 for various doses of TLC388HCl

| | Pharmacokinetic Parameter, Mean (SD) | | | |
|---|---|---|---|---|
| Dose Group | AUC₀₋ₜ | Cₘₐₓ | Tₘₐₓ | t_{1/2} |
| (mg/m²) | (hr · ng/mL) | (ng/mL) | (hr) | (hr) |
| 1.5 | 2.872 (0.882) | 2.117 (0.468) | 0.505 (0.039) | 1.953 (0.698) |
| 3.0 | 5.625 (1.972) | 4.600 (2.102) | 0.467 (0.209) | 2.686 (1.501) |
| 6.0 | 11.599 (1.437) | 11.483 (5.651) | 0.483 (0.000) | 2.252 (0.261) |
| 9.0 | 16.581 (3.976) | 9.605 (4.201) | 0.529 (0.240) | 2.750 (0.530) |
| 13.5 | 20.677 (6.804) | 19.675 (2.394) | 0.292 (0.277) | 1.626 (0.927) |
| 20.0 | 29.023 (7.443) | 25.800 (7.766) | 0.405 (0.135) | 2.161 (0.225) |
| 30.0 | 78.761 (50.88) | 81.400 (93.80) | 0.512 (0.240) | 2.258 (0.454) |
| 35.0 | 102.97 (20.18) | 74.800 (42.82) | 0.592 (0.091) | 3.347 (1.219) |
| 40.0 | 72.758 (49.22) | 104.39 (188.6) | 0.440 (0.175) | 1.864 (0.595) |
| 50.0 | 97.314 (38.34) | 67.060 (31.13) | 0.417 (0.155) | 2.478 (0.408) |
| 60.0 | 109.80 (25.72) | 80.320 (14.05) | 0.450 (0.115) | 2.371 (0.385) |

The pK profiles of Topotecan in Day 1 and Day 8 are shown in Table 14 and Table 15, respectively.

TABLE 14
Day 1 pK profiles of Topotecan for various doses of TLC388HCl

| | Pharmacokinetic Parameter, Mean (SD) | | | | |
|---|---|---|---|---|---|
| Dose Group | AUC₀₋ₜ | Cₘₐₓ | | Tₘₐₓ | t_{1/2} |
| (mg/m²) | (hr · ng/mL) | (ng/mL) | | (hr) | (hr) |
| 1.5 | 6.006 (1.206) | 1.520 | (0.466) | 0.628 (0.068) | 4.761 (0.834) |
| 3.0 | 11.731 (7.179) | 2.103 | (0.742) | 1.222 (0.694) | 7.030 (2.954) |
| 6.0 | 21.500 (6.845) | 4.830 | (2.274) | 0.989 (0.876) | 6.873 (1.934) |
| 9.0 | 27.631 (15.09) | 6.380 | (1.709) | 1.150 (0.722) | 7.700 (2.307) |
| 13.5 | 37.616 (7.148) | 8.713 | (0.727) | 0.629 (0.249) | 6.294 (3.269) |
| 20.0 | 47.309 (13.02) | 11.313 | (4.077) | 0.483 (0.000) | 5.072 (1.436) |
| 30.0 | 97.035 (34.62) | 21.225 | (3.960) | 1.537 (1.650) | 4.056 (0.760) |
| 35.0 | 175.74 (44.94) | 51.325 | (39.31) | 1.000 (0.667) | 7.659 (2.185) |
| 40.0 | 141.35 (41.35) | 29.740 | (12.73) | 0.850 (1.112) | 5.838 (2.358) |
| 50.0 | 207.88 (84.54) | 37.520 | (14.65) | 0.903 (0.650) | 7.386 (6.088) |
| 60.0 | 215.13 (60.94) | 57.080 | (29.20) | 0.837 (0.655) | 5.177 (1.636) |

TABLE 15
Day 8 pK profiles of Topotecan for various doses of TLC388HCl

| | Pharmacokinetic Parameter, Mean (SD) | | | | | |
|---|---|---|---|---|---|---|
| Dose Group | AUC₀₋ₜ | | Cₘₐₓ | | Tₘₐₓ | t_{1/2} |
| (mg/m²) | (hr · ng/mL) | | (ng/mL) | | (hr) | (hr) |
| 1.3 | 5.316 | (1.688) | 1.063 | (0.330) | 0.906 (0.518) | 4.324 (0.679) |
| 3.0 | 7.866 | (3.556) | 1.770 | (0.425) | 0.544 (0.106) | 8.216 (0.143) |
| 6.0 | 21.656 | (6.352) | 5.070 | (1.652) | 0.600 (0.202) | 5.202 (1.747) |
| 9.0 | 37.978 | (16.81) | 6.210 | (2.206) | 1.829 (1.586) | 12.267 (5.026) |
| 13.5 | 30.971 | (17.27) | 9.425 | (2.195) | 1.408 (1.744) | 3.601 (1.594) |
| 20.0 | 44.026 | (11.66) | 10.447 | (2.223) | 0.405 (0.135) | 4.647 (1.033) |
| 30.0 | 120.74 | (59.93) | 48.575 | (58.41) | 1.450 (1.708) | 4.635 (1.550) |
| 35.0 | 190.91 | (74.13) | 58.075 | (22.75) | 0.604 (0.156) | 6.177 (3.376) |
| 40.0 | 497.96 | (1255) | 3713.6 | (11661) | 1.032 (1.141) | 6.380 (1.360) |
| 50.0 | 207.30 | (93.53) | 40.940 | (22.20) | 0.857 (0.598) | 5.515 (1.056) |
| 60.0 | 207.43 | (81.42) | 69.280 | (44.75) | 0.566 (0.152) | 4.967 (2.321) |

TABLE 16
Dose normalized Cₘₐₓ, AUC, Tₘₐₓ and T_{1/2} for TLC-388HCl diastereomers (i.e.,
S,R-TLC388, S,S-TLC388) and three metabolites: TPT (topotecan), TLC-U1 and TLC-U2
in patients treated with 60 mg/m² of TLC388HCl (N = 6).

| | S,R-TLC388 | S,S-TLC388 | TPT | TLC-U1 | TLC-U2 |
|---|---|---|---|---|---|
| Dose-normalized | 1.63 ± 3.77 | 2.60 ± 6.02 | 1.95 ± 1.04 | 10.15 ± 3.87 | 2.84 ± 0.97 |
| Cₘₐₓ | | | | | |
| (nM/(mg/m²)) | | | | | |
| Dose-normalized | 0.82 ± 1.92 | 1.22 ± 2.87 | 8.52 ± 3.09 | 14.49 ± 4.46 | 4.05 ± 1.34 |
| AUCₗₐₛₜ | | | | | |
| (nM*h/(mg/m²)) | | | | | |
| tₘₐₓ (h) | 0.47 ± 0.30 | 0.47 ± 0.3 | 0.93 ± 0.82 | 0.45 ± 0.17 | 0.47 ± 0.14 |
| t_{1/2} (h) | 1.08 ± 1.77 | 0.75 ± 1.18 | 6.05 ± 2.71 | 3.13 ± 1.27 | 2.58 ± 0.62 |

2. Summary of the TLC388HCl pK Profile:

Now referring to FIG. 2 and Table 16, after the IV administration of TLC388HCl composition, the peak plasma concentrations (Cₘₐₓ) of S,R-TLC388, S,S-TLC388, TLC-U1, TLC-U2 and topotecan were achieved within one hour. The plasma concentration of TLC388HCl diastereomers (i.e., S,R-TLC388, S,S-TLC388) fell quickly after that, indicating a short half-life (a few minutes) and a rapid metabolism and clearance. This is followed by a rapid rise (within 15 minutes of starting the infusion) of the three metabolites in the serum: TLC-U1, TLC-U2 and topotecan. The plasma concentrations of the three metabolites were higher than that of TLC388HCl within one hour of the IV Lipotecan® infusion.

As shown in Table 16, the peak concentration (Cₘₐₓ) and area under the concentration-time cure (AUC) of S,S-TLC388HCl were higher than those of S,R-TLC388HCl at all dose levels, as S,S and S, R diastereomers were in approximately a 2:1 ratio.

On the other hand, S,S-TLC388HCl and S,R-TLC388HCl took the same amount of time to reach peak concentrations (Tₘₐₓ) at all dose levels.

At all dose levels, the Cₘₐₓ and AUCₒ₋ₜ of TLC-U1 were approximately 3-4 times greater than those of TLC-U2 and approximately 2 times greater than those of topotecan. The Cₘₐₓ and AUCₒ₋ₜ of TLC-U1 were also higher than those of S,S-TLC388 and S,R-TLC388, indicating that the patients had highest exposure to TLC-U1.

The Tₘₐₓ of S,S-TLC388, S,R-TLC388 and its metabolites, TLC-U1 and TLC-U2, was less than 1 hour at all doses and occurred near the end of infusion (30 minutes). At all dose levels, the three metabolites, TLC-U1, TLC-U2 and topotecan, showed longer T_{1/2} (>2.5 hours) and higher plasma concentrations when compared with the parent compounds, S,S- and S,R-TLC388HCl. Tₘₐₓ for Topotecan was slightly longer than the other two metabolites, indicating that S,S-TLC388 and S,R-TLC388 were rapidly metabolized into TLC-U1 and TLC-U2, respectively, which were then metabolized into the active Topotecan. FIGS. 2 and 5 illustrate that the TLC388HCl composition provides a sustained release of TLC-U1, TLC-U2 and topotecan over a period of at least about 8 hours in subjects treated with 60 mg/m² (FIG. 2) or 1.5 mg/m² (FIG. 5) TLC388HCl. At the same dose level, Cₘₐₓ and AUC of TLC-U1 were higher than those of TLC-U2 and topotecan, indicating that patients had maximum exposure to TLC-U1.

There is no significant difference between the PK profiles of S,R-TLC388HCl, S,S-TLC388HCl, TLC-U1 TLC-U2 and topotecan on Day 1 and Day 8 of Cycle 1. As the two doses were given 7 days apart, which is equivalent to 8 folds of T_{1/2}, there was no accumulation of TLC388HCl at the time of second dose administration (Day 8 of Cycle 1).

3. Summary of Anti-Cancer Efficacy:

Twenty of 36 evaluable patients had stable disease (SD) after 2 cycles of treatment.

Remission (≧6 months with stable disease) or minor response was noted in 9 patients with the following types of cancer: renal, salivary gland, vagina and thymoma. Some of these patients were refractory to other chemotherapy agents prior to joining the trial.

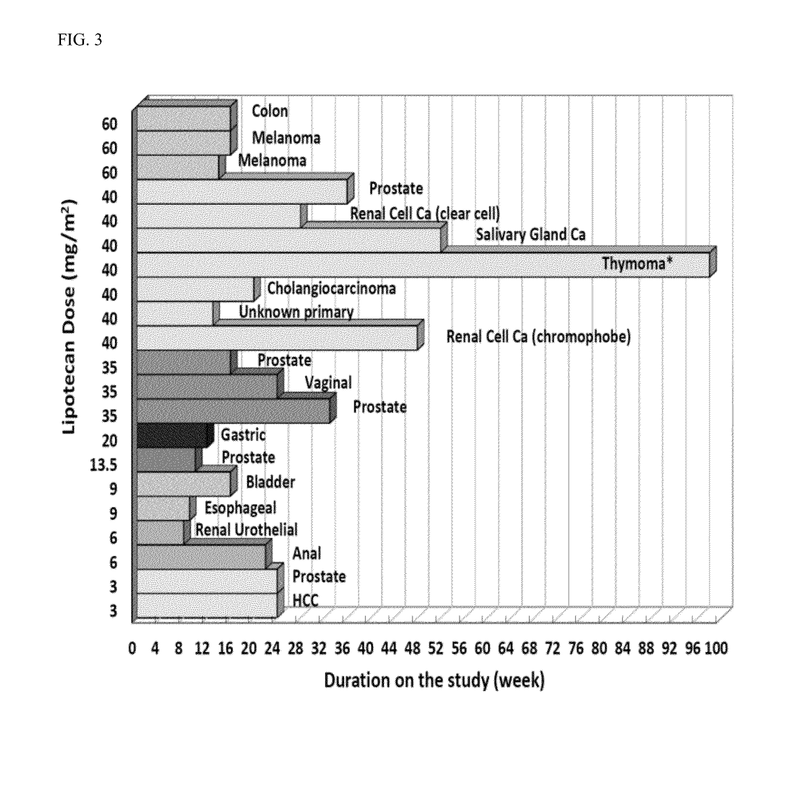

FIG. 3 shows the type of cancer, doses of TLC388HCl composition and duration of treatment in patients with stable disease. 35 mg/m² and 40 mg/m² dosages were most efficacious in inhibiting cancer cells growth, as patients have the longer duration of treatment.

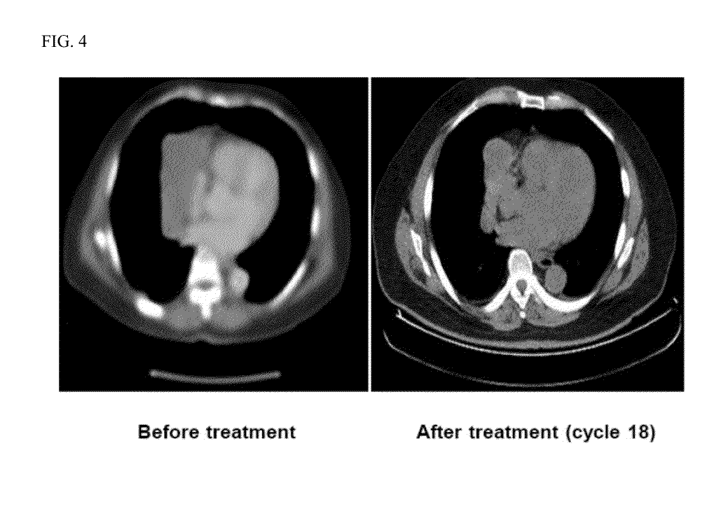

One outstanding anti-cancer response was observed in a 70-year-old male with stage II B thymoma and metastasis to lung, liver and lymph nodes. He was refractory to other types of chemotherapy agents (cyclophosphamide, cisplatin and doxorubicin). However, he had a remarkable response to TLC388HCl composition and was subsequently treated with 20 cycles. FIG. 4 is the CT images before and after 18 cycles of TLC388HCl treatment, which shows a reduction of the tumor size by 20%.

Summary:

One embodiment of the present invention, TLC388 composition, was an effective single anti-cancer agent and showed a favorable safety profile in patients with cancer. Extended therapy did not lead to cumulative toxicity. Based on the safety profile, the maximum tolerated dose of TLC388HCl is about 50 mg/m².

The mean PK data suggested that exposure to TLC388, TLC-U1, TLC-U2 and topotecan was roughly dose proportional. TLC388HCl composition provides a sustained release of TLC-U1, TLC-U2 and topotecan over a period of at least about 8 hours. There was no evidence of drug accumulation, evidenced by Cₘₐₓ and AUC values of Day 1 and Day 8.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A method for inhibiting cancer cell growth in a subject, said method comprising the step of:
administering to said subject a composition comprising:
at least one compound of formula (I): 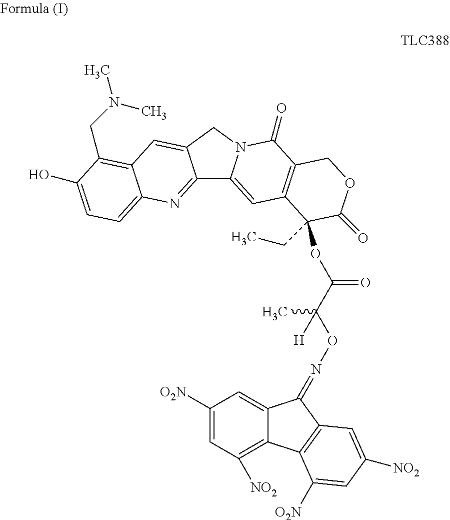 [SMILES: CC[C@@]1(OC(=O)C(C)ON=C2c3cc([N+](=O)[O-])cc([N+](=O)[O-])c3-c3c2cc([N+](=O)[O-])cc3[N+](=O)[O-])C(=O)OCc2c1cc1n(c2=O)Cc2cc3c(CN(C)C)c(O)ccc3nc2-1]
or a pharmaceutically acceptable salt thereof; and
at least one polyethylene glycol (PEG) conjugated phospholipid;
wherein the molar ratio of said PEG conjugated phospholipid to said compound or said pharmaceutically acceptable salt of said compound is greater than about 0.45:1;
wherein said composition provides a sustained release of a therapeutically effective amount of topotecan to said subject over a period of at least about 8 hours.

2. The method of claim 1,
wherein said pharmaceutically acceptable salt of said compound is TLC388HCl.

3. The method of claim 1,
wherein said compound is S,S-TLC388, S,R-TLC388, or a mixture thereof.

4. The method of claim 3,
wherein said compound is a diastereomeric mixture of S,S-TLC388 and S,R-TLC388 in a molar ratio of about 2:1 (S,S-TLC388: S,R-TLC388).

5. The method of claim 1,
wherein said composition is administered at a level of 1.5 mg/m² to about 60 mg/m².

6. The method of claim 1,
wherein said composition is administered at a level of greater than about 40 mg/m².

7. The method of claim 1,
wherein said administering step is repeated weekly.

8. The method of claim 1,
wherein the Cₘₐₓ of said topotecan is about 1 ng/mL to about 3720 ng/mL.

9. The method of claim 1,
wherein the Cₘₐₓ of said topotecan is about 25 ng/mL to about 3720 ng/mL.

10. The method of claim 1,
wherein the tₘₐₓ of said topotecan is about 0.4 hours to about 1.9 hours.

11. The method of claim 1,
wherein the tₘₐₓ of said topotecan is about 0.8 hours to about 1.0 hours.

12. The method of claim 1,
wherein the AUC₀₋₈ₕᵣ of said topotecan is about 5 hr-ng/mL to about 500 hr-ng/mL.

13. The method of claim 1,
wherein the AUC₀₋₈ₕᵣ of said topotecan is about 140 hr-ng/mL to about 500 hr-ng/mL.

14. The method of claim 1,
wherein the t_{1/2} of said topotecan is about 4 hours to about 12.5 hours.

15. The method of claim 1,
wherein the t_{1/2} of said topotecan is about 4.5 hours to about 7.5 hours.

16. The method of claim 1,
wherein said composition further provides a sustained release of TLC-U2 to said subject over a period of at least about 8 hours.

17. The method of claim 16,
wherein the Cₘₐₓ of said TLC-U2 is about 2 ng/mL to about 105 ng/mL.

18. The method of claim 16,
wherein the Cₘₐₓ of said TLC-U2 is about 50 ng/mL to about 110 ng/mL.

19. The method of claim 16,
wherein the tₘₐₓ of said TLC-U2 is about 0.2 hours to about 0.7 hours.

20. The method of claim 16,
wherein the tₘₐₓ of said TLC-U2 is about 0.3 hours to about 0.5 hours.

21. The method of claim 16,
wherein the AUC₀₋₈ₕᵣ of said TLC-U2 is about 2.5 hr-ng/mL to about 115 hr-ng/mL.

22. The method of claim 16,
wherein the AUC₀₋₈ₕᵣ of said TLC-U2 is about 70 hr-ng/mL to about 115 hr-ng/mL.

23. The method of claim 16,
wherein the t_{1/2} of said TLC-U2 is about 1.5 hours to about 3.5 hours.

24. The method of claim 16,
wherein the t_{1/2} of said TLC-U2 is about 1.5 hours to about 3.0 hours.

25. The method of claim 1,
wherein said composition further provides a sustained release of TLC-U1 to said subject over a period of at least about 8 hours.

26. The method of claim 25,
wherein the Cₘₐₓ of said TLC-U1 is about 5 ng/mL to about 450 ng/mL.

27. The method of claim 25,
wherein the Cₘₐₓ of said TLC-U1 is about 200 ng/mL to about 450 ng/mL.

28. The method of claim 25,
wherein the tₘₐₓ of said TLC-U1 is about 0.2 hours to about 0.7 hours.

29. The method of claim 25,
wherein the tₘₐₓ of said TLC-U1 is about 0.3 hours to about 0.6 hours.

30. The method of claim 25,
wherein the AUC₀₋₈ₕᵣ of said TLC-U1 is about 5 hr-ng/mL to about 430 hr-ng/mL.

31. The method of claim 25,
wherein the AUC₀₋₈ₕᵣ of said TLC-U1 is about 270 hr-ng/mL to about 430 hr-ng/mL.

32. The method of claim 25,
wherein the t_{1/2} of said TLC-U1 is about 1.5 hours to about 5.6 hours.

33. The method of claim 25,
wherein the t_{1/2} of said TLC-U1 is about 1.8 hours to about 3.5 hours.

34. The method of claim 1,
wherein said composition further comprises at least one pH adjusting agent.

35. The method of claim 1,
wherein the molar ratio of said PEG conjugated phospholipid to said compound or pharmaceutically acceptable salt of said compound is about 0.60:1 to about 1.00:1.

36. The method of claim 1,
wherein the molar ratio of said PEG conjugated phospholipid to said compound or pharmaceutically acceptable salt of said compound is about 0.70:1 to about 0.90:1.

37. The method of claim 1,
wherein said composition has a pH less than about 4.

38. The method of claim 1,
wherein said PEG conjugated phospholipid comprises a PEG moiety having a molecular weight from about 1,000 to about 20,000 daltons.

39. The method of claim 1,
wherein said PEG conjugated phospholipid is a PEG-DSPE (distearoyl-phosphatidylethanolamine) conjugate.

40. The method of claim 39,
wherein the PEG-DSPE conjugate is a methoxyl PEG-DSPE conjugate.

41. The method of claim 1, wherein the composition is administered as a single dose.
